(19) United States
(12) Patent Application Publication
(10) Pub. No.: US 2022/0125852 A1
(43) Pub. Date: Apr. 28, 2022

(54) PROTECTION AND REGENERATION OF NEUROLOGICAL FUNCTION BY USING STEM CELLS

(71) Applicant: Therapeutic Solutions International, Inc., Oceanside, CA (US)

(72) Inventors: Thomas E. Ichim, Oceanside, CA (US); Timothy G. Dixon, Oceanside, CA (US); Amit N. Patel, Miami, FL (US); Famela Ramos, Oceanside, CA (US); Kalina O'Connor, Oceanside, CA (US)

(73) Assignee: Therapeutic Solutions International, Inc., Oceanside, CA (US)

(21) Appl. No.: 17/512,496

(22) Filed: Oct. 27, 2021

Related U.S. Application Data
(60) Provisional application No. 63/105,964, filed on Oct. 27, 2020.

(51) Int. Cl. A61K 35/28

(52) U.S. Cl. CPC A61K 35/28

(57) ABSTRACT

Disclosed are therapeutic compounds, protocols, and compositions of matter useful for treatment of neurological conditions. In one embodiment the invention teaches the treatment of chronic traumatic encephalopathy (CTE) through protecting/regenerating the endothelial by administration of cells such as stem cells. In one embodiment stem cells are administered in order to protect the endothelium from apoptosis and to preserve the blood brain barrier. In another embodiment stem cells are administered together with endothelial progenitor cells in order to regenerate neural endothelium. In other embodiments preservation of brain integrity in conditions of degeneration is accomplished by administration of stem cells and/or endothelial cells.

### CROSS-REFERENCE TO RELATED APPLICATIONS

This application claims the benefit of priority to U.S. Provisional Application No. 63/105,964, filed Oct. 27, 2020, the contents of which are hereby incorporated by reference.

### FIELD OF THE INVENTION

The invention pertains to the use of stem cells for protecting and regenerating neurological function. The teachings herein are useful for treatment of conditions such as chronic traumatic encephalopathy and schizophrenia.

### BACKGROUND

Modern day study of CTE was publicized by the pioneer work of pathologist Bennett Omalu, when in 2005 he reported a representative case of a retired National Football (NFL) player with progressive neurological dysfunction [6]. According to Omalu, the term CTE includes dementia pugilistica and supplants the use of the term dementia pugilistica. Due to initial controversy, and implications of CTE on various sports, it has been said that CTE is a very peculiar condition, in part because according to some authors “it is unique among brain diseases in having a history of decades of organized opposition to its codification as an authentic or valid entity [7].”

About one-third of CTE cases are progressive, but clinical progression is not always sequential or predictable. The clinical symptoms vary extensively, which is probably due to multiple damage sites among athletes with the condition [8]. The severity varies from mild complaints, to severe deficits accompanied by dementia, Parkinson-like symptoms, and behavioral changes. Clinical symptoms include neurological and cognitive complaints together with psychiatric and behavioral disturbances. Early neurological symptoms may include speech problems and impaired balance, while later symptoms include ataxia, spasticity, impaired coordination, and extrapyramidal symptoms, with slowness of movements and tremor [8, 9]. Cognitive problems, such as attention deficits and memory disturbances, often become major factors in later stages of the disease, although may occur at varying times throughout the course of CTE. Psychiatric and behavioral problems include lack of insight and judgment, depression, disinhibition, euphoria, hypomania, irritability, aggressiveness and suicidal tendencies.

CTE is also unique because it is typically defined after the patient dies, based on autopsy examination of the brain. According to more recent criteria, there are 4 stages of CTE, all with increasing neuropathology [4, 8, 10-15].

In Stage 1 CTE, at the macroscopic level, the brain appears normal, however, immunohistochemistry reveals the presence of phosphorylated tau in a limited number of places in the brain, usually in lateral and frontal cortices, as well as proximal to small blood vessels in the depth of sulci. Although unclear, it is believed at a clinical level, patients with Stage 1 CTE appear generally asymptomatic, or in some situations exhibit short term memory deficiency In some cases mild depression and/or concurrent aggressiveness is exhibited.

In State 2 CTE, there are some distinct anatomical deviations that may be seen such as enlargement of lateral ventricles, cavum septum pellucidum with or without fenestration, as well as pallor of the locus coeruleus and substantia nigra. Using immunohistochemistry, depositions of phosphorylated tau can be seen deep in the sulci, and there is an emergent spreading pattern. Behaviorally, Stage 2 CTE is characterized by mood and behavioral symptoms which could include behavioral outbursts and more severe depressive symptoms.

In Stage 3 CTE, macroscopic abnormalities are highly visible. Also, global brain weight loss, mild frontal lobe and temporal lobe atrophy, and dilation of the ventricles is observed. In these patients, one half display septal abnormalities, including cavum septum pellucidum. Furthermore, immunocytochemistry reveals that tau pathology spreads, involving the frontal, temporal, parietal and insular cortices. At a clinical level these patients present with more cognitive deficits, including memory loss, executive functioning deficits, visuospatial dysfunction, and apathy.

In Stage 4 CTE, there is a major reduction in brain weight, with brains weighing up to 30% less than control brains when “age-matched”. Severe atrophy of the frontal, medial temporal lobes, as well as anterior thalami is observed, along with atrophy of the white matter tracts. The majority of Stage 4 patients have septal abnormalities. The spread of the p-tau affects most regions, including the calcarine cortex. At a clinical level, patients present with advanced language deficits, psychotic symptoms which include paranoia, motor deficits, and parkinsonism.

### SUMMARY

The teachings herein include methods of preserving integrity of the blood brain barrier comprising: a) obtaining a patient at risk of blood brain barrier leakage, and/or already having leakage of said blood brain barrier; b) administering to said patient one or more cellular populations; c) assessing said patient and when necessary adjusting dose of said cellular populations.

Further embodiments include methods wherein said blood brain barrier is a selective barrier that separates circulating blood from the brain.

Further embodiments include methods wherein said blood brain barrier is comprised of endothelial cells bound together by tight junction proteins that form the blood facing side of the lumen of the small cerebral blood vessels.

Further embodiments include methods wherein astrocytes (in particular, projections from those cells termed astrocytic feet) and pericytes contribute to the structure and function of said blood brain barrier.

Further embodiments include methods wherein said patient having a risk of blood brain barrier leakage, and/or of already having blood brain barrier leakage suffers from a neurological condition.

Further embodiments include methods wherein said neurological condition is selected from a group comprising of Abulia, Achromatopsia, Agraphia, AIDS—neurological manifestations, Akinetopsia, Alcoholism, Alien hand syndrome, Allan-Herndon-Dudley syndrome, Alternating hemiplegia of childhood, Alzheimer's disease, Amaurosis fugax, Amnesia, Amyotrophic lateral sclerosis, Aneurysm, Angelman syndrome, Anosognosia, Aphasia, Aphantasia, Apraxia, Arachnoiditis, Arnold-Chiari malformation, Asomatognosia, Asperger syndrome, Ataxia, ATR-16 syndrome, Attention deficit hyperactivity disorder, Auditory processing disorder, Autism spectrum disorder, Behçet's disease, Bell's palsy, Bipolar disorder, Blindsight, Brachial plexus injury, Brain injury, Brain tumor, Brody myopathy, Canavan disease, Capgras delusion, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy, Cerebral dysgenesis-neuropathy-ichthyosis-keratoderma syndrome, Cerebral gigantism, Cerebral palsy, Cerebral vasculitis, Cerebrospinal fluid leak, Cervical spinal stenosis, Charcot-Marie-Tooth disease, Chiari malformation, Chorea, Chronic fatigue syndrome, Chronic inflammatory demyelinating polyneuropathy, Chronic pain, Cluster Headache, Cockayne syndrome, Coffin-Lowry syndrome, Coma, Complex regional pain syndrome, Compression neuropathy, Congenital distal spinal muscular atrophy, Congenital facial diplegia, Corticobasal degeneration, Cranial arteritis, Craniosynostosis, Creutzfeldt-Jakob disease, Cumulative trauma disorders, Cushing's syndrome, Cyclic vomiting syndrome, Cyclothymic disorder, Cytomegalic inclusion body disease, Cytomegalovirus Infection, Dandy-Walker syndrome, Dawson disease, De Morsier's syndrome, Dejerine-Klumpke palsy, Dejerine-Sottas disease, Delayed sleep phase disorder or syndrome, Dementia, Dermatomyositis, Developmental coordination disorder, Diabetic neuropathy, Diffuse sclerosis, Diplopia, Disorders of consciousness, Distal hereditary motor neuropathy type V, Distal spinal muscular atrophy type 1, Distal spinal muscular atrophy type 2, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalopathy, Encephalotrigeminal angiomatosis, Encopresis, Enuresis, Epilepsy, Epilepsy-intellectual disability in females, Erb's palsy, Erythromelalgia, Essential tremor, Exploding head syndrome, Fabry's disease, Fahr's syndrome, Fainting, Familial spastic paralysis, Fetal alcohol syndrome, Febrile seizures, Fisher syndrome, Fibromyalgia, Foville's syndrome, Fragile X syndrome, Fragile X-associated tremor/ataxia syndrome, Friedreich's ataxia, Frontotemporal dementia, Functional neurological symptom disorder, Gaucher's disease, Generalized anxiety disorder, Generalized epilepsy with febrile seizures plus, Gerstmann's syndrome, Giant cell arteritis, Giant cell inclusion disease, Globoid cell leukodystrophy, Gray matter heterotopia, Guillain-Barré syndrome, Head injury, Headache, Hemicrania Continua, Hemifacial spasm, Hemispatial neglect, Hereditary motor neuropathies, Hereditary motor neuropathies, Hereditary spastic paraplegia, Heredopathia atactica polyneuritiformis, Herpes zoster, Herpes zoster oticus, Hirayama syndrome, Hirschsprung's disease, Holmes-Adie syndrome, Holoprosencephaly, HTLV-1 associated myelopathy, Huntington's disease, Hydranencephaly, Hydrocephalus, Hypercortisolism, Hypoalgesia, Hypoesthesia, cerebral hypoxia, Immune-mediated encephalomyelitis, Inclusion body myositis, Incontinentia pigmenti, Refsum disease, Infantile spasms, Inflammatory myopathy, Intracranial cyst, Intracranial hypertension, Joubert syndrome, Karak syndrome, Kearns-Sayre syndrome, Kinsbourne syndrome, Kleine-Levin syndrome, Klippel Feil syndrome, Krabbe disease, Kufor-Rakeb syndrome, Kugelberg-Welander disease, Lafora disease, Lambert-Eaton myasthenic syndrome, Landau-Kleffner syndrome, Lateral medullary (Wallenberg) syndrome, Leigh's disease, Lennox-Gastaut syndrome, Lesch-Nyhan syndrome, Leukodystrophy, Leukoencephalopathy with vanishing white matter, Lewy body dementia, Lissencephaly, Locked-in syndrome, Lupus erythematosus-neurological sequelae, Lyme disease, Machado-Joseph disease, Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Morvan syndrome, Motor skills disorder, Moyamoya disease, Mucopolysaccharidoses, Multifocal motor neuropathy, Multi-infarct dementia, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotonia congenita, Myotubular myopathy, Narcolepsy, Neuro-Behçet's disease, Neurofibromatosis, Neuroleptic malignant syndrome, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, Occipital Neuralgia, Occult spinal dysraphism sequence, Ohtahara syndrome, Olivopontocerebellar atrophy, Opsoclonus myoclonus syndrome, Optic neuritis, Orthostatic hypotension, O'Sullivan-McLeod syndrome, Otosclerosis, Palinopsia, PANDAS, Pantothenate kinase-associated neurodegeneration, Paramyotonia congenita, Paresthesia, Parkinson's disease, Paraneoplastic diseases, Paroxysmal attacks, Parry-Romberg syndrome, Pelizaeus-Merzbacher disease, Periodic paralyses, Peripheral neuropathy, Pervasive developmental disorders, Phantom limb/Phantom pain, Photic sneeze reflex, Phytanic acid storage disease, Pick's disease, Pinched nerve, Pituitary tumors, polyneuropathy, PMG, Polio, Polymicrogyria, Polymyositis, Porencephaly, Post-polio syndrome, Postherpetic neuralgia, Posttraumatic stress disorder, Postural hypotension, Postural orthostatic tachycardia syndrome, Prader-Willi syndrome, Primary lateral sclerosis, Prion diseases, Progressive hemifacial atrophy, Progressive multifocal leukoencephalopathy, Progressive supranuclear palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III—see Ramsay-Hunt syndrome, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic movement disorder, Romberg syndrome, Saint Vitus dance, Sandhoff disease, Sanfilippo syndrome, Schilder's disease (two distinct conditions), Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal and bulbar muscular atrophy, Spinal cord injury, Spinal cord tumors, Spinal muscular atrophy, Spinal muscular atrophy with respiratory distress type 1, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tardive dyskinesia, Tarlov cyst, Tarsal tunnel syndrome, Tay-Sachs disease, Temporal arteritis, Temporal lobe epilepsy, Tetanus, Tethered spinal cord syndrome, Thalamocortical dysrhythmia, Thomsen disease, Thoracic outlet syndrome, Tic Douloureux, Tinnitus, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Vestibular schwannoma, Viliuisk encephalomyelitis, Visual Snow, Von Hippel-Lindau disease, Wallenberg's syndrome, Werdnig-Hoffmann disease, Wernicke's encephalopathy, West syndrome, Williams syndrome, Wilson's disease, Y-Linked hearing impairment, and Zellweger syndrome

Further embodiments include methods wherein said cellular population is a mesenchymal stem cell.

Further embodiments include methods wherein said mesenchymal stem cell is plastic adherent.

Further embodiments include methods wherein said mesenchymal stem cell is CD7 positive.

Further embodiments include methods wherein said mesenchymal stem cell is interleukin 1 receptor positive.

Further embodiments include methods wherein said mesenchymal stem cell is interleukin 3 receptor positive.

Further embodiments include methods wherein said mesenchymal stem cell is interleukin 6 receptor positive.

Further embodiments include methods wherein said mesenchymal stem cell is interleukin 13 receptor positive.

Further embodiments include methods wherein said mesenchymal stem cell is interleukin 17 receptor positive.

Further embodiments include methods wherein said mesenchymal stem cell is interleukin 17F receptor positive.

Further embodiments include methods wherein said mesenchymal stem cell is interleukin 10 receptor positive.

Further embodiments include methods wherein said mesenchymal stem cell is CD11 positive.

Further embodiments include methods wherein said mesenchymal stem cell is CD90 positive.

Further embodiments include methods wherein said mesenchymal stem cell is CD105 positive.

Further embodiments include methods wherein said mesenchymal stem cell is CD133 positive.

Further embodiments include methods wherein said mesenchymal stem cells are derived from bone marrow.

Further embodiments include methods wherein said mesenchymal stem cells are derived from placenta.

Further embodiments include methods wherein said mesenchymal stem cells are derived from peripheral blood.

Further embodiments include methods wherein said mesenchymal stem cells are derived from menstrual blood.

Further embodiments include methods wherein said mesenchymal stem cells are derived from adipose tissue.

Further embodiments include methods wherein said mesenchymal stem cells are derived from Wharton's Jelly.

Further embodiments include methods wherein said mesenchymal stem cells are derived from umbilical cord.

Further embodiments include methods wherein said mesenchymal stem cells are derived from fallopian tube.

### BRIEF DESCRIPTION OF THE DRAWINGS

FIG. 1 is a bar graph showing Conditioned media (CM) from JadiCells decreases endothelial cell death when HUVEC cells are cultured with upstream inflammatory agent TNF-alpha.

FIG. 2 is a bar graph showing Conditioned media (CM) from JadiCells decreases endothelial cell death when or HUVEC cells are cultured with downstream H2O2.

FIG. 3 is a bar graph showing HLA expression on HUVEC was utilized to quantify endothelial antigen presentation.

FIG. 4 is a bar graph showing mixed lymphocyte reaction used as a test of T cell activation.

FIG. 5 is a bar graph showing results of HUVEC cells treated with endotoxin to stimulate activation, where an assessment of Tissue Factor was performed by flow cytometry.

### DETAILED DESCRIPTION OF THE INVENTION

The invention provides the repair of blood brain barrier by administration of mesenchymal stem cells. The invention describes a previously unknown effect of mesenchymal stem cells to: a) protect endothelium from oxidative stress; b) reduce inflammation induced endothelial antigen presenting function; and c) suppress inflammation induced endothelial thrombogenicity.

In some embodiments of the invention, mesenchymal stem cells are utilized to treat chronic traumatic encephalopathy (CTE).

For use in the invention, numerous types of mesenchymal stem cells may be used. For the practice of the invention, MSCs can be used together with complement inhibition for the purpose of immune modulation. The invention discloses that MSC may be viewed as a “intelligent” immune modulators. In contrast to current therapies, which globally cause immune suppression, production of anti-inflammatory factors by MSC appears to be dependent on their environment, with upregulation of factors such as TGF-b, HLA-G, IL-10, and neuropilin-A ligands galectin-1 and Semaphorin-3A in response to immune/inflammatory stimuli but little in the basal state [499-503]. This property may be selected for when utilizing the marker combinations disclosed in the current invention. Additionally, the invention discloses synergies between complement inhibition and MSC administration of induction of immune modulation and/or tolerogenesis. The combined use of MSC and complement inhibition may be directed towards conditions such as autoimmunity, transplant rejection, inflammation, sepsis, ARDS and acute radiation syndrome.

Additionally, systemically administered MSC possess ability to selectively home to injured/hypoxic areas by recognition of signals such as HMGB 1 or CXCR1, respectively [504-507]. The ability to home to injury, combined with selective induction of immune modulation only in response to inflammatory/danger signals suggests the possibility that systemically administered MSC do not cause global immune suppression. This is supported by clinical studies using MSC for other inflammatory conditions, which to date, have not reported immune suppression associated adverse effects [508-510]. Another important aspect of MSC therapy is their ability to regenerate injured tissue through direct differentiation into articular tissue [511], as well as ability to secret growth factors capable of augmenting endogenous regenerative processes [512].

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like

In one embodiment MSC donor lots are generated from umbilical cord tissue. Means of generating umbilical cord tissue MSC have been previously published and are incorporated by reference [513-519]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

In one embodiment, bone marrow MSC lots are generated, means of generating BM MSC are known in the literature and examples are incorporated by reference.

In one embodiment BM-MSC are generated as follows

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

A concussion is a type of traumatic brain injury which affects brain function. These effects are usually temporary but can include headaches and problems with concentration, memory, balance and coordination. Concussions are usually caused by a blow to the head. Violently shaking the head and upper body also can cause concussions. In some concussions the patient loses consciousness, but most do not. It's possible to have a concussion and not realize it [5, 16, 17].

Concussion has been recognized as a clinical entity for more than 1,000 years. Throughout the 20th century it was studied extensively in boxers, but it did not pique the interest of the general population because it is the accepted goal of the boxer to inflict such an injury on their opponent. In 2002, however, the possibility that repetitive concussions could result in chronic brain damage and a progressive neurologic disorder was raised by a postmortem evaluation of a retired player in the most popular sports institution in the United States, the National Football League. Since that time, concussion has been a frequent topic of conversation in homes, schools, and throughout the media It has become a major focus of sports programs in communities and schools at all levels [16].

Concussive injuries are also a problem in the military and industrial worksites. In the case of the former, traumatic brain injury resulting from exposure to the force of a detonation trigger, similar neuropathological mechanisms leading to neuropathology and sequelae indistinguishable to chronic traumatic encephalopathy is observed. In some cases, concussion causes no gross pathology, such as hemorrhage, and no abnormalities on structural brain imaging. There also may be no loss of consciousness. Many other complaints such as dizziness, nausea, reduced attention and concentration, memory problems, and headache have been reported. A greater likelihood of unconsciousness occurs with more severe concussions. These types of concussive head impacts are very frequent in American football whose athletes, especially linemen and linebackers, may be exposed to more than 1,000 impacts per season [18]. The effects of multiple concussions are becoming better recognized in these professional athletes, but much less is known about the long term-effects of repeated concussion in the brains of amateur athletes, teenagers and adolescents. Moreover, the amateur codes of football are less regulated than the professional codes, and the adolescent brain may be more vulnerable to concussion. The better-developed neck musculature of the professional football player, the more strictly controlled tackling and the better aftercare of the concussed professional means that the long-term public health problem of concussion in sport is grossly underestimated.

Military personnel who have experienced concussion experience a range of detrimental and chronic medical conditions. Concussion occurring among soldiers deployed in Iraq is strongly associated with Post Traumatic Stress Disease (PTSD) and physical health problems 3 to 4 months after the soldiers return home. PTSD and depression are important mediators of the relationship between mild traumatic brain injury and physical health problems. PTSD was strongly associated with mild traumatic brain injury. It was reported that overall, 43.9% of soldiers who reported loss of consciousness met the criteria for PTSD, as compared with 27.3% of those with altered mental status, 16.2% of those with other injuries, and 9.1% of those with no injuries [19]. Also, more than 1 in 3 returning military troops who have sustained a deployment-related concussion have headaches which meet criteria for post traumatic headache [20]. It has been shown that nearly 15% of combat personnel sustained concussion while on duty [19]. Repeated concussion is a serious issue for combat personnel. A study showed that a majority of concussion incidents were blast related. The median time between events was 40 days, with 20% experiencing a second event within 2 weeks of the first and 87% within 3 months [21].

While an isolated concussion has been widely considered to be an innocuous event, recent studies [9, 12] have suggested that repeated concussion is associated with the development of a neurodegenerative disorder known as chronic traumatic encephalopathy (CTE). CTE is regarded as a disorder which often occurs in midlife, years or decades after the sports or military career has ended [5, 8, 12]. It is believed that in England at least 17% of boxers have CTE as judged by disturbed gait and coordination, slurred speech and tremors, as well as cerebral dysfunction causing cognitive impairments and neurobehavioural disturbances [22]. In one study, diffusion tensor imaging (DTI), which is sensitive to microscopic white matter changes when routine MR imaging is unrevealing [23, 24], was used together with tract-based spatial statistics (TBSS) together with neuropsychological examination of executive functions and memory to investigate a collective of 31 male amateur boxers and 31 age-matched controls as well as a subgroup of 19 individuals, respectively, who were additionally matched for intellectual performance (IQ). It was found that participants had normal findings in neurological examination and conventional MR. Amateur boxers did not show deficits in neuropsychological tests when their IQ was taken into account. Fractional anisotropy was significantly reduced, while diffusivity measures were increased along central white matter tracts in the boxers' group. These changes were in part associated with the number of fights. This study demonstrated that TBSS revealed widespread white matter disturbance partially related to the individual fighting history in amateur boxers. These findings closely resemble those in patients with accidental TBI and indicate similar histological changes in amateur boxers [25].

In addition to boxing, Jockeys have also been reported to suffer from CTE, in a 1976 publication, Foster et al reported Five National Hunt jockeys have been found to have post-traumatic encephalopathy—three with epilepsy and two with significant intellectual and psychological deterioration [26]. Other reports of jockey's having similar situations have been described [27]. Numerous other causes of CTE have been described including whiplash [28], shaken baby syndrome [29], wrestling [30], military combat [31, 32], football [6, 33-36], rugby [37], soccer [38, 39], jail head trauma [40], shotgun injury [41] and mixed martial arts [42].

One study in the Journal of the American Medical Association (JAMA) examined a case series of 202 football players whose brains were donated for research. Neuropathological evaluations and retrospective telephone clinical assessments (including head trauma history) with informants were performed blinded. Online questionnaires ascertained athletic and military history. Neuropathological diagnoses of neurodegenerative diseases, including CTE, was based on defined diagnostic criteria. These included CTE neuropathological severity (stages I to IV or dichotomized into mild [stages I and II] and severe [stages III and IV]); informant-reported athletic history and, for players who died in 2014 or later, clinical presentation, including behavior, mood, and cognitive symptoms and dementia. Among 202 deceased former football players (median age at death, 66 years [interquartile range, 47-76 years]), CTE was neuropathologically diagnosed in 177 players (87%; median age at death, 67 years [interquartile range, 52-77 years]; mean years of football participation, 15.1 [SD, 5.2]), including 0 of 2 pre-high school, 3 of 14 high school (21%), 48 of 53 college (91%), 9 of 14 semiprofessional (64%), 7 of 8 Canadian Football League (88%), and 110 of 111 National Football League (99%) players. Neuropathological severity of CTE was distributed across the highest level of play, with all 3 former high school players having mild pathology and the majority of former college (27 [56%]), semiprofessional (5 [56%]), and professional (101 [86%]) players having severe pathology. Among 27 participants with mild CTE pathology, 26 (96%) had behavioral or mood symptoms or both, 23 (85%) had cognitive symptoms, and 9 (33%) had signs of dementia. Among 84 participants with severe CTE pathology, 75 (89%) had behavioral or mood symptoms or both, 80 (95%) had cognitive symptoms, and 71 (85%) had signs of dementia. In a sample of deceased football players who donated their brains for research, a high proportion had neuropathological evidence of CTE, suggesting that CTE may be related to prior participation in football [43].

In another study, the authors examined the effect of age of first exposure to tackle football on chronic traumatic encephalopathy (CTE) pathological severity and age of neurobehavioral symptom onset in tackle football players with neuropathologically confirmed CTE. The sample included 246 tackle football players who donated their brains for neuropathological examination. Two hundred eleven were diagnosed with CTE (126 of 211 were without comorbid neurodegenerative diseases), and 35 were without CTE. Informant interviews ascertained age of first exposure and age of cognitive and behavioral/mood symptom onset. Analyses accounted for decade and duration of play. Age of exposure was not associated with CTE pathological severity, Alzheimer's disease or Lewy body pathology. In the 211 participants with CTE, every 1-year younger participants began to play tackle football predicted earlier reported cognitive symptom onset by 2.44 years (p<0.0001) and behavioral/mood symptoms by 2.50 years (p<0.0001). Age of exposure before 12 predicted earlier cognitive (p<0.0001) and behavioral/mood (p<0.0001) symptom onset by 13.39 and 13.28 years, respectively. In participants with dementia, the younger age of exposure corresponded to earlier functional impairment onset. Similar effects were observed in the 126 CTE-only participants. Effect sizes were comparable in participants without CTE. In this sample of deceased football players, the younger age of exposure to tackle football was not associated with CTE pathological severity, but predicted earlier neurobehavioral symptom onset. Youth exposure to football may reduce resiliency to late-life neuropathology [44].

One of the major observations found in patients with CTE is the extensive presence of neurofibrillary tangles [8, 12, 45-48]. Tangles are found intracellularly in the cytoplasm of neurons and consist of threadlike aggregates of hyperphosphorylated tau protein. In some cases, peripheral levels of Tau are reported to be elevated [49]. Tau is a normal axonal protein that binds to microtubules via their microtubule binding domains, thus promoting microtubule assembly and stability [50-54].

The hyperphosphorylated form of tau causes disassembly of microtubules and thus impaired axonal transport, leading to compromised neuronal and synaptic function, increased propensity of tau aggregation, and subsequent formation of insoluble fibrils and tangles [55, 56]. Unlike in Alzheimer's disease, tangles in athletes with CTE tend to accumulate perivascularly within the superficial neocortical layers, particularly at the base of the sulci. Tau pathology in CTE is also patchy and irregularly distributed, possibly related to the many different directions of mechanical force induced by physical trauma [12]. It is the accumulation of hyperphosphorylated tau protein that is thought to result in the development of CTE and its associated psychiatric and behavioral disturbances.

How does tau become hyperphosphorylated in CTE? One hypothesis is that brain damage is associated with activation of caspase-3, which cleaves tau in a manner predisposing it to phosphorylation, as well as taking abnormal and potentially pathological confirmations [45, 57, 58]. Another proposed mechanism relates to decreased alkaline phosphatase that occurs as a result of various head injuries. For example, in one study, researchers used blast or weight drop models of traumatic brain injury (TBI) in rats, and observed pTau accumulation in the brain as early as 6 hours post-injury and further accumulation which varied regionally by 24 h post-injury. The pTau accumulation was accompanied by reduced tissue non-specific alkaline phosphatase (TNAP) expression, activity in the injured brain regions and a significantly decreased plasma total alkaline phosphatase activity after the weight drop. These results reveal that both blast- and impact acceleration-induced head injuries cause an acute decrease in the level/activity of TNAP in the brain, which potentially contributes to trauma-induced accumulation of pTau and the resultant tauopathy. The regional changes in the level/activity of TNAP or accumulation of pTau after these injuries did not correlate with the accumulation of amyloid precursor protein, suggesting that the basic mechanism underlying tauopathy in TBI might be distinct from that associated with AD [59].

One of the interesting properties of the tau associated pathology is what appears to be the ability of phosphorylated tau to “spread” throughout the brain in a manner that has been previously compared to prion disease. One of the first possibilities of this type of tau propagation was suggested in a brain study. The brain extracted from deceased individuals with PiD, a neurodegenerative disorder characterized by three-repeat (3R) tau prions, were used to infect HEK293T cells expressing 3R tau fused to yellow fluorescent protein (YFP). Extracts from patient samples, which contain four-repeat (4R) tau prions, were transmitted to HEK293 cells expressing 4R tau fused to YFP. These studies demonstrated that prion propagation in HEK cells requires isoform pairing between the infecting prion and the recipient substrate. Interestingly, tau aggregates in AD and CTE, containing both 3R and 4R isoforms, were unable to robustly infect either 3R- or 4R-expressing cells. However, AD and CTE prions were able to replicate in HEK293T cells expressing both 3R and 4R tau.

Unexpectedly, increasing the level of 4R isoform expression alone supported the propagation of both AD and CTE prions. These results allowed us to determine the levels of tau prions in AD and CTE brain extracts [60]. In a more definitive animal study, scientists evaluated whether moderate to severe TBI can trigger the initial formation of pathological tau that would induce the development of the pathology throughout the brain. To this end, the authors subjected tau transgenic mice to TBI and assessed tau phosphorylation and aggregation pattern to create a spatial heat map of tau deposition and spreading in the brain. The results suggest that brain injured tau transgenic mice have an accelerated tau pathology in different brain regions that increases over time compared to sham mice. The appearance of pathological tau occurs in regions distant to the injury area which are synaptically connected, indicating the spreading of tau aggregates spreading in a prion-like manner [61].

A more comprehensive study examined the ability of aggregated tau to spread. Scientists demonstrated a single severe brain trauma is associated with the emergence of widespread hyperphosphorylated tau pathology in a proportion of humans surviving after injury. In parallel experimental studies, a model of severe traumatic brain injury in wild-type mice, found progressive and widespread tau pathology, replicating the findings in humans. Brain homogenates from these mice, when inoculated into the hippocampus and overlying cerebral cortex of naïve mice, induced widespread tau pathology, synaptic loss, and persistent memory deficits. Accordingly, this data provides evidence that experimental brain trauma induces a self-propagating tau pathology, which can be transmitted between mice, and call for future studies aimed at investigating the potential transmissibility of trauma associated tau pathology in humans [62]. The ability of the pathological tau to spread has been postulated as one of the mechanisms by which brain pathology advances in patients with CTE years, if not decades, after cessation of repetitive injuries [63]. Interestingly some studies have demonstrated the possibility of using antibodies to inhibit pathological tau [64].

It is widely known that one result of a head injury is inflammation. However, the concept of propagating inflammation and self-maintaining inflammation is something relatively new. In contrast to traditional TBI, in which there is one major acute insult, CTE is characterized by multiple smaller insults, and in some cases progression of pathology increases despite large periods of time during after which the damaging agent has been removed.

One of the cardinal features of CTE, which initiates with the concussive or subconcussive brain injury is the activation of the microglia. The microglia cells are brain residing macrophage lineage cells whose main physiological function is the phagocytosis of debris, as well as protection of the CNS from various pathogens. In one study, immunohistochemistry for reactive microglia (CD68 and CR3/43) was performed on human autopsy brain tissue and assessed ‘blind’ by quantitative image analysis. Head injury cases were compared with age matched controls, and within the traumatic brain injury group cases with diffuse traumatic axonal injury were compared with cases without diffuse traumatic axonal injury. The study found a neuroinflammatory response that develops within the first week and persists for several months after traumatic brain injury [65]. In a CTE study, the effects of repetitive head impacts (RHI) on the development of neuroinflammation and its relationship to CTE where examined. Specifically, the investigation aimed to determine the relationship between RHI exposure, neuroinflammation, and the development of hyperphosphorylated tau (pTau) pathology and dementia risk in CTE.

A cohort of 66 deceased American football athletes from the Boston University-Veteran's Affairs-Concussion Legacy Foundation Brain Bank as well as 16 non-athlete controls where utilized for the investigation. Subjects with a neurodegenerative disease other than CTE were excluded. Counts of total and activated microglia, astrocytes, and phosphorylated tau pathology were performed in the dorsolateral frontal cortex (DLF). Binary logistic and simultaneous equation regression models were used to test associations between RHI exposure, microglia, pTau pathology, and dementia. Duration of RHI exposure and the development and severity of CTE were associated with reactive microglial morphology and increased numbers of CD68 immunoreactive microglia in the DLF. A simultaneous equation regression model demonstrated that RHI exposure had a significant direct effect on CD68 cell density (p<0.0001) and pTau pathology (p<0.0001) independent of age at death. The effect of RHI on pTau pathology was partially mediated through increased CD68 positive cell density. A binary logistic regression demonstrated that a diagnosis of dementia was significantly predicted by CD68 cell density (OR=1.010, p=0.011) independent of age (OR=1.055, p=0.007), but this effect disappeared when pTau pathology was included in the model. In conclusion, RHI is associated with chronic activation of microglia, which may partially mediate the effect of RHI on the development of pTau pathology and dementia in CTE. The authors concluded that inflammatory molecules may be important diagnostic or predictive biomarkers as well as promising therapeutic targets in CTE [66].

It is known that activated microglia produce kynurenine, in part through upregulation of the enzyme indolamine 2,3, deoxygenase [67-71]. An imbalance of neuroactive kynurenine pathway metabolites has been proposed as one mechanism behind the neuropsychiatric sequelae of certain neurological disorders. It has been hypothesized that concussed football players would have elevated plasma levels of neurotoxic kynurenine metabolites and reduced levels of neuroprotective metabolites relative to healthy football players and that altered kynurenine levels would correlate with post-concussion mood symptoms. In one study, Mood scales and plasma concentrations of kynurenine metabolites were assessed in concussed (N=18; 1.61 days post-injury) and healthy football players (N=18). A subset of football players returned at 1-week (N=14; 9.29 days) and 1-month post-concussion (N=14, 30.93 days).

Concussed athletes had significantly elevated levels of quinolinic acid (QUIN) and significantly lower ratios of kynurenic acid (KYNA) to QUIN at all time points compared with healthy athletes (p's<0.05), with no longitudinal evidence of normalization of KYNA or KYNA/QUIN. At 1-day post-injury, concussed athletes with lower levels of the putatively neuroprotective KYNA/QUIN ratio reported significantly worse depressive symptoms (p=0.04), and a trend toward worse anxiety symptoms (p=0.06), while at 1-month higher QUIN levels were associated with worse mood symptoms (p's<0.01). Finally, concussed athletes with worse concussion outcome, defined as number of days until return-to-play, had higher QUIN and lower KYNA/QUIN at 1-month post-injury (p's<0.05). The authors concluded that the results converge with existing kynurenine literature on psychiatric patients and provide the first evidence of altered peripheral levels of kynurenine metabolites following sports-related concussion [72].

Direct monitoring of brain inflammation in vivo has been reported in a pilot study in which former National Football League (NFL) players were examined by new neuroimaging techniques and clinical measures of cognitive functioning. It was hypothesized that former NFL players would show molecular and structural changes in medial temporal and parietal lobe structures as well as specific cognitive deficits, namely those of verbal learning and memory. A significant increase in binding of [(11)C]DPA-713 to the translocator protein (TSPO), a marker of brain injury and repair, in several brain regions, such as the supramarginal gyms and right amygdala, in 9 former NFL players compared to 9 age-matched, healthy controls was observed. Additionally, significant atrophy of the right hippocampus was seen. Finally, these same former players had varied performance on a test of verbal learning and memory, suggesting that these molecular and pathologic changes may play a role in cognitive decline. These results suggest that localized brain injury and repair, indicated by increased [(11)C]DPA-713 binding to TSPO, may be linked to history of NFL play. [(11)C]DPA-713 PET is a promising new tool that can be used in future study design to examine further the relationship between TSPO expression in brain injury and repair, selective regional brain atrophy, and the potential link to deficits in verbal learning and memory after NFL play [73].

### Examples

### JadiCell Conditioned Media Inhibits Endothelial Death.

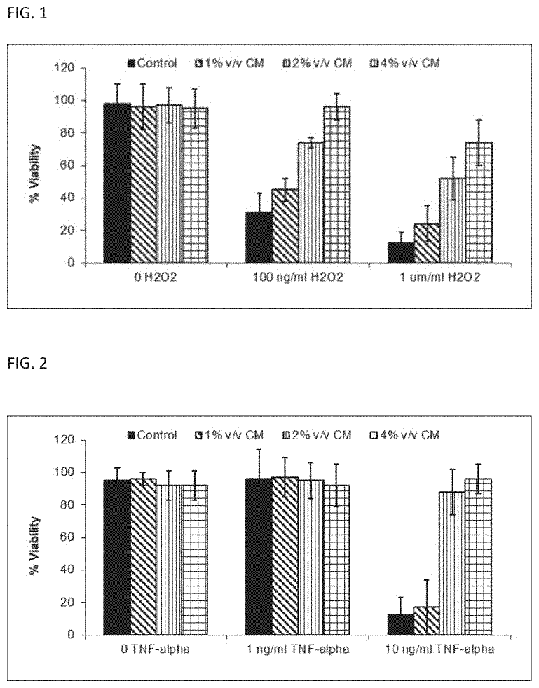

Injury associated with inflammation. Inflammation causes oxidative stress. Oxidative stress induces death of endothelial cells. Endothelial death exposes basement membrane, induces coagulopathy. Culture of HUVEC cells with upstream inflammatory agent TNF-alpha or downstream H₂O₂ results in death. Conditioned media (CM) from JadiCells decreases endothelial cell death. Results are shown in FIGS. 1 and 2.

### JadiCell™ Reduces Immunogenicity of Endothelial Cells

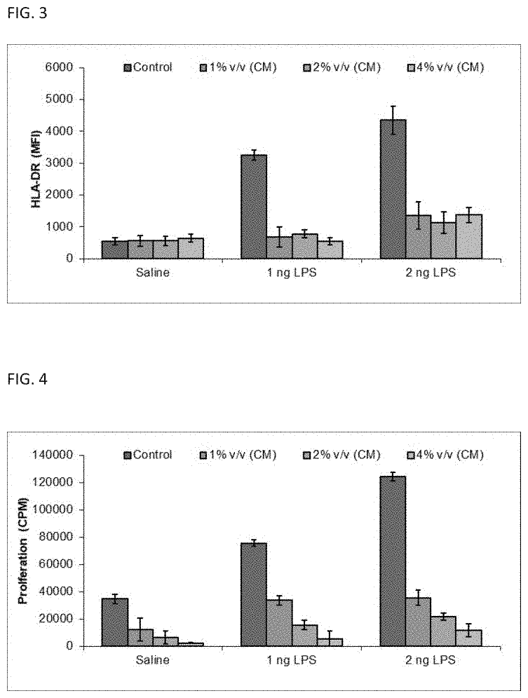

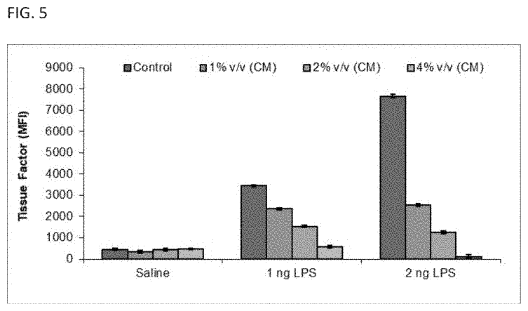

Endothelial cells can act as antigen presenting cells. Stimulation of T cells by endothelial cells results in inflammation and breaking of blood brain barrier. HLA expression on HUVEC was utilized to quantify one aspects of endothelial antigen presentation. Mixed lymphocyte reaction used as a test of T cell activation. Results are shown in FIGS. 3 and 4.

### JadiCell™ Reduces Thrombogenicity of Activated Endothelial Cells

In response to inflammation endothelial cells induce clotting by stimulation of extrinsic coagulation pathway. Tissue factor is activator of extrinsic pathway. HUVEC cells were treated with endotoxin to stimulate activation. Assessment of Tissue Factor performed by flow cytometry. Results are shown in FIG. 5.

1. A method preserving integrity of the blood brain barrier comprising: a) obtaining a patient at risk of blood brain barrier leakage, and/or already having leakage of said blood brain barrier; b) administering to said patient one or more cellular populations; c) assessing said patient and when necessary adjusting dose of said cellular populations.

2. The method of claim 1, wherein said blood brain barrier is a selective barrier that separates circulating blood from the brain.

3. The method of claim 2, wherein said blood brain barrier is comprised of endothelial cells bound together by tight junction proteins that form the blood facing side of the lumen of the small cerebral blood vessels.

4. The method of claim 3, wherein astrocytes (in particular, projections from those cells termed astrocytic feet) and pericytes contribute to the structure and function of said blood brain barrier.

5. The method of claim 1, wherein said patient having a risk of blood brain barrier leakage, and/or of already having blood brain barrier leakage suffers from a neurological condition.

6. The method of claim 5, wherein said neurological condition is selected from a group comprising of Abulia, Achromatopsia, Agraphia, AIDS—neurological manifestations, Akinetopsia, Alcoholism, Alien hand syndrome, Allan-Herndon-Dudley syndrome, Alternating hemiplegia of childhood, Alzheimer's disease, Amaurosis fugax, Amnesia, Amyotrophic lateral sclerosis, Aneurysm, Angelman syndrome, Anosognosia, Aphasia, Aphantasia, Apraxia, Arachnoiditis, Arnold-Chiari malformation, Asomatognosia, Asperger syndrome, Ataxia, ATR-16 syndrome, Attention deficit hyperactivity disorder, Auditory processing disorder, Autism spectrum disorder, Behçet's disease, Bell's palsy, Bipolar disorder, Blindsight, Brachial plexus injury, Brain injury, Brain tumor, Brody myopathy, Canavan disease, Capgras delusion, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy, Cerebral dysgenesis-neuropathy-ichthyosis-keratoderma syndrome, Cerebral gigantism, Cerebral palsy, Cerebral vasculitis, Cerebrospinal fluid leak, Cervical spinal stenosis, Charcot-Marie-Tooth disease, Chiari malformation, Chorea, Chronic fatigue syndrome, Chronic inflammatory demyelinating polyneuropathy, Chronic pain, Cluster Headache, Cockayne syndrome, Coffin-Lowry syndrome, Coma, Complex regional pain syndrome, Compression neuropathy, Congenital distal spinal muscular atrophy, Congenital facial diplegia, Corticobasal degeneration, Cranial arteritis, Craniosynostosis, Creutzfeldt-Jakob disease, Cumulative trauma disorders, Cushing's syndrome, Cyclic vomiting syndrome, Cyclothymic disorder, Cytomegalic inclusion body disease, Cytomegalovirus Infection, Dandy-Walker syndrome, Dawson disease, De Morsier's syndrome, Dejerine-Klumpke palsy, Dejerine-Sottas disease, Delayed sleep phase disorder or syndrome, Dementia, Dermatomyositis, Developmental coordination disorder, Diabetic neuropathy, Diffuse sclerosis, Diplopia, Disorders of consciousness, Distal hereditary motor neuropathy type V, Distal spinal muscular atrophy type 1, Distal spinal muscular atrophy type 2, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalopathy, Encephalotrigeminal angiomatosis, Encopresis, Enuresis, Epilepsy, Epilepsy-intellectual disability in females, Erb's palsy, Erythromelalgia, Essential tremor, Exploding head syndrome, Fabry's disease, Fahr's syndrome, Fainting, Familial spastic paralysis, Fetal alcohol syndrome, Febrile seizures, Fisher syndrome, Fibromyalgia, Foville's syndrome, Fragile X syndrome, Fragile X-associated tremor/ataxia syndrome, Friedreich's ataxia, Frontotemporal dementia, Functional neurological symptom disorder, Gaucher's disease, Generalized anxiety disorder, Generalized epilepsy with febrile seizures plus, Gerstmann's syndrome, Giant cell arteritis, Giant cell inclusion disease, Globoid cell leukodystrophy, Gray matter heterotopia, Guillain-Barré syndrome, Head injury, Headache, Hemicrania Continua, Hemifacial spasm, Hemispatial neglect, Hereditary motor neuropathies, Hereditary motor neuropathies, Hereditary spastic paraplegia, Heredopathia atactica polyneuritiformis, Herpes zoster, Herpes zoster oticus, Hirayama syndrome, Hirschsprung's disease, Holmes-Adie syndrome, Holoprosencephaly, HTLV-1 associated myelopathy, Huntington's disease, Hydranencephaly, Hydrocephalus, Hypercortisolism, Hypoalgesia, Hypoesthesia, cerebral hypoxia, Immune-mediated encephalomyelitis, Inclusion body myositis, Incontinentia pigmenti, Refsum disease, Infantile spasms, Inflammatory myopathy, Intracranial cyst, Intracranial hypertension, Joubert syndrome, Karak syndrome, Kearns-Sayre syndrome, Kinsbourne syndrome, Kleine-Levin syndrome, Klippel Feil syndrome, Krabbe disease, Kufor-Rakeb syndrome, Kugelberg-Welander disease, Lafora disease, Lambert-Eaton myasthenic syndrome, Landau-Kleffner syndrome, Lateral medullary (Wallenberg) syndrome, Leigh's disease, Lennox-Gastaut syndrome, Lesch-Nyhan syndrome, Leukodystrophy, Leukoencephalopathy with vanishing white matter, Lewy body dementia, Lissencephaly, Locked-in syndrome, Lupus erythematosus-neurological sequelae, Lyme disease, Machado-Joseph disease, Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Morvan syndrome, Motor skills disorder, Moyamoya disease, Mucopolysaccharidoses, Multifocal motor neuropathy, Multi-infarct dementia, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotonia congenita, Myotubular myopathy, Narcolepsy, Neuro-Behçet's disease, Neurofibromatosis, Neuroleptic malignant syndrome, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, Occipital Neuralgia, Occult spinal dysraphism sequence, Ohtahara syndrome, Olivopontocerebellar atrophy, Opsoclonus myoclonus syndrome, Optic neuritis, Orthostatic hypotension, O'Sullivan-McLeod syndrome, Otosclerosis, Palinopsia, PANDAS, Pantothenate kinase-associated neurodegeneration, Paramyotonia congenita, Paresthesia, Parkinson's disease, Paraneoplastic diseases, Paroxysmal attacks, Parry-Romberg syndrome, Pelizaeus-Merzbacher disease, Periodic paralyses, Peripheral neuropathy, Pervasive developmental disorders, Phantom limb/Phantom pain, Photic sneeze reflex, Phytanic acid storage disease, Pick's disease, Pinched nerve, Pituitary tumors, polyneuropathy, PMG, Polio, Polymicrogyria, Polymyositis, Porencephaly, Post-polio syndrome, Postherpetic neuralgia, Posttraumatic stress disorder, Postural hypotension, Postural orthostatic tachycardia syndrome, Prader-Willi syndrome, Primary lateral sclerosis, Prion diseases, Progressive hemifacial atrophy, Progressive multifocal leukoencephalopathy, Progressive supranuclear palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III—see Ramsay-Hunt syndrome, Rasmussen encephalitis, Reflex neurovascular dystrophy, Refsum disease, REM sleep behavior disorder, Repetitive stress injury, Restless legs syndrome, Retrovirus associated myelopathy, Rett syndrome, Reye's syndrome, Rhythmic movement disorder, Romberg syndrome, Saint Vitus dance, Sandhoff disease, Sanfilippo syndrome, Schilder's disease (two distinct conditions), Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal and bulbar muscular atrophy, Spinal cord injury, Spinal cord tumors, Spinal muscular atrophy, Spinal muscular atrophy with respiratory distress type 1, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tardive dyskinesia, Tarlov cyst, Tarsal tunnel syndrome, Tay-Sachs disease, Temporal arteritis, Temporal lobe epilepsy, Tetanus, Tethered spinal cord syndrome, Thalamocortical dysrhythmia, Thomsen disease, Thoracic outlet syndrome, Tic Douloureux, Tinnitus, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Vestibular schwannoma, Viliuisk encephalomyelitis, Visual Snow, Von Hippel-Lindau disease, Wallenberg's syndrome, Werdnig-Hoffmann disease, Wernicke's encephalopathy, West syndrome, Williams syndrome, Wilson's disease, Y-Linked hearing impairment, and Zellweger syndrome

7. The method of claim 1, wherein said cellular population is a mesenchymal stem cell.

8. The method of claim 7, wherein said mesenchymal stem cell is plastic adherent.

9. The method of claim 7, wherein said mesenchymal stem cell is CD7 positive.

10. The method of claim 7, wherein said mesenchymal stem cell is interleukin 1 receptor positive.

11. The method of claim 7, wherein said mesenchymal stem cell is interleukin 3 receptor positive.

12. The method of claim 7, wherein said mesenchymal stem cell is interleukin 6 receptor positive.

13. The method of claim 7, wherein said mesenchymal stem cell is interleukin 13 receptor positive.

14. The method of claim 7, wherein said mesenchymal stem cell is interleukin 17 receptor positive.

15. The method of claim 7, wherein said mesenchymal stem cell is interleukin 17F receptor positive.

16. The method of claim 7, wherein said mesenchymal stem cell is interleukin 10 receptor positive.

17. The method of claim 7, wherein said mesenchymal stem cell is CD11 positive.

18. The method of claim 7, wherein said mesenchymal stem cell is CD90 positive.

19. The method of claim 7, wherein said mesenchymal stem cell is CD105 positive.

20. The method of claim 7, wherein said mesenchymal stem cell is CD133 positive.
